(19) United States
(12) Patent Application Publication
(10) Pub. No.: US 2022/0202698 A1
(43) Pub. Date: Jun. 30, 2022

(54) EXTENDED RELEASE PHARMACEUTICAL COMPOSITIONS OF RIOCIGUAT

(71) Applicant: Jubilant Pharma Holdings Inc., Yardley, PA (US)

(72) Inventors: Indranil Nandi, Yardley, PA (US); Tusharmouli Mukherjee, Yardley, PA (US); Dinesh Kumar, Noida (IN); Rakesh Kumar Singh, Noida (IN)

(73) Assignee: Jubilant Pharma Holdings Inc., Yardley, PA (US)

(21) Appl. No.: 17/562,487

(22) Filed: Dec. 27, 2021

Related U.S. Application Data
(60) Provisional application No. 63/130,973, filed on Dec. 28, 2020.

(51) Int. Cl. A61K 9/00; A61K 9/20; A61K 31/4162

(52) U.S. Cl. CPC A61K 9/0002; A61K 31/4162; A61K 9/2027; A61K 9/2054

(57) ABSTRACT

Provided herein are the extended release pharmaceutical composition suitable for once or twice daily dosing comprising riociguat and at least one or more pharmaceutically acceptable excipients. The present invention also relates to method for preparing extended release composition and method of using these dosage forms for the treatment of pulmonary hypertension and related diseases. The present invention provides extended release composition of riociguat which are expected to exhibit desired technical attributes such as assay, stability and release profile suitable for once or twice daily administration.

### FIELD OF THE INVENTION

The present invention relates to extended release pharmaceutical compositions of riociguat suitable for once or twice daily administration. The present invention also relates to methods for preparing extended release dosage forms. The prepared dosage forms are suitable for the treatment of pulmonary hypertension and related diseases.

### BACKGROUND OF THE INVENTION

Riociguat is an antihypertensive drug. It is chemically known as methyl 4,6-diamino-2-[1-(2-fluorobenzyl)-1H-pyrazolo[3,4-b]pyridin-3-yl]-5-pyrimidinyl(methyl)carbamate and is represented by the following formula as:

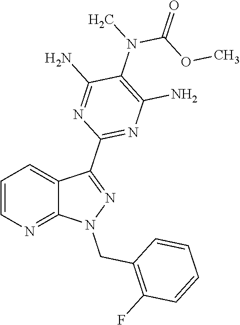 [SMILES: COC(=O)N(C)c1c(N)nc(-c2nn(Cc3ccccc3F)c3ncccc23)nc1N]

Riociguat is marketed in the U.S. as an immediate release tablet dosage form in 0.5 mg, 1 mg, 1.5 mg, 2 mg and 2.5 mg strengths under the brand name Adempas® by Bayer Healthcare. The marketed dosage form of riociguat is indicated for the treatment of persistent/recurrent chronic thromboembolic pulmonary hypertension (CTEPH) and pulmonary arterial hypertension (PAH) to improve exercise capacity.

U.S. Pat. No. 7,173,037, assigned to Bayer Healthcare, discloses riociguat as a new compound. This patent also discloses the use of riociguat in the treatment of hypertension.

U.S. Pat. Nos. 10,087,183 and 10,662,188, assigned to Bayer Healthcare, discloses various polymorphs of riociguat such as modification I, modification II, mono-DMSO solvate, sesqui-DMSO solvate, ¼-ethyl acetate solvate and pharmaceutical composition thereof.

Indian publication no. 201921012816, assigned to Cipla, discloses extended release pharmaceutical formulation comprising riociguat, at least one release controlling agent in combination with a swelling agent, and at least one pharmaceutically acceptable excipient. The release controlling agent is present in the examples at about 45% w/w to 60% w/w. Notably, Examples 1-3 of the patent publication have between 44-49% release controlling agent (with ratio of riociguat to release controlling agent of about 1:2 to about 1:2.3) yet have very different dissolution profiles, demonstrating the unpredictability of making a dosage form of riociguat with an extended release profile. The patent publication specifically suggests the use of polyethylene oxide of desired molecular weight and grade as swelling agents in combination of cellulose derivatives. The said publication fails to disclose any glidant free extended release composition of riociguat.

Riociguat is a BCS Class II compound and accordingly has low solubility and high permeability. Riociguat is rapidly absorbed after oral administration, the maximum concentration in plasma (Cₘₐₓ) being reached after approximately 0.8-1.0 hr. with riociguat 0.5-2.5 mg administered as an immediate release oral tablet in healthy male volunteers. Therefore, the commercially available immediate release pharmaceutical formulations of riociguat are recommended to administer as starting dosage of 1 mg taken 3 times a day. Riociguat has an elimination half-life of about 12 hours. Use of an immediate release oral dosage form in the treatment of hypertension may not be a suitable approach due to number of factors such as frequent dosage administration, long treatment period and low patient compliance. To facilitate patient compliance, there is a need to design extended release dosage form of riociguat for a once or twice daily formulation.

Extended or sustained release compositions can offer clinically significant advantages by increasing patient compliance due to reduced frequency of administration, improved safety and efficacy of drug and reductio of undesirable side-effects in comparison to the corresponding immediate release dosage form. No marketed extended release formulations of riociguat are known so far; it is understood that once or twice daily extended release dosage form disclosure are yet away from practical application in the treatment of pulmonary hypertension.

The inventors of the present invention have developed extended release pharmaceutical compositions of riociguat with an objective to eliminate frequent dosing administration, which is likely to improve patient compliance and accordingly provide a more convenient and less cumbersome posology. Further, extended release pharmaceutical compositions of riociguat are expected to exhibit desirable technical attributes such as stability, assay and a release profile suitable for once or twice daily dosing.

Therefore, in view of the advantages associated with extended release formulations, it is desirable to develop extended release formulations of riociguat suitable for once or twice daily administration. There is also an unmet need to provide an alternate dosage form, which would provide higher compliance rate along with maintaining therapeutic levels of the drug in the patient's system for long period of time for the treatment of pulmonary hypertension and related disorders.

### SUMMARY OF THE INVENTION

It is an object of the present invention to provide an extended release pharmaceutical composition suitable for once or twice daily dosing comprising riociguat and at least one or more pharmaceutically acceptable excipients.

The present invention also relates to extended release compositions of riociguat and at least one or more pharmaceutically acceptable excipients and processes for preparing such compositions.

The present invention also relates to extended release compositions comprising riociguat and at least one or more pharmaceutically acceptable excipients selected from the group comprising diluent, binder, disintegrant, controlled release polymer, antioxidant, lubricant, glidant, surfactant, plasticizer, wetting agent, solubilizer, stabilizer, anticaking agent, antifoaming agent, alkaline agent, film-forming polymer, opacifier and coloring agent.

The present invention also relates to the use of extended release compositions of riociguat in the manufacture of medicament for the treatment of hypertension, pulmonary hypertension, pulmonary arterial hypertension, chronic thromboembolic pulmonary hypertension, heart failure, arrhythmia, high blood pressure, angina pectoris, myocardial infarction, stroke, transient ischaemic attacks, arteriosclerosis, erectile dysfunction, osteoporosis, alzheimer's disease, parkinson's disease, multiple sclerosis, depression, schizophrenia, bipolar disorder and migraine.

### DETAILED DESCRIPTION OF THE INVENTION

The present invention can be more readily understood by reading the following detailed description of the invention and study of the included examples.

As used herein, the term “composition”, “formulation”, “dosage form”, is intended to encompass a drug product comprising riociguat, and other inert ingredient(s) (pharmaceutically acceptable excipients). Such pharmaceutical compositions are synonymous with “formulation”, “dosage form”, “extended release composition”, “controlled release composition”, “modified release composition”, “sustained release composition”, “prolonged release composition”, “delayed release composition”, “programmed release composition”, “timed release composition” and/or “rate controlled” compositions or dosage forms and are used synonymously throughout the application. Pharmaceutical compositions of the invention include, but are not limited to, multiparticulates, pellets, beads, spherules, beadlets, microcapsules, millispheres, microspheres, powder, granules, spheroids and the like, tablets, capsules, sprinkles, sachet, inlay tablets, matrix tablet, osmotic tablet, MuPs tablet, tablet-in-tablet, mini-tablet, micro-tablet, tablet-in-capsule, single layer and bilayer tablet, trilayer tablet, capsule fill comprises any alone or combination of immediate-release, extended-release and delayed-release components or like. The pharmaceutical compositions of present invention are suitable for at least once daily administration. Solid oral dosage forms are preferably selected from the group comprising of tablets with a modified release matrix, functionally coated tablets, capsules, mini tablets, pellets and granules. In accordance with other embodiment, riociguat is the sole active ingredient in the extended release dosage form.

The term “core” as used herein is defined as the pharmaceutical composition without coating. The phrase “inert core,” as used herein, includes a core that may be microcrystalline cellulose spheres, sugar spheres, non-pariel seeds, dibasic calcium phosphate bead, mannitol bead, silica bead, tartaric acid pellet, wax based pellet and water insoluble and non-swellable beads. The term “multiparticulate” as used herein refers to a plurality of discrete or aggregated particles, pellets, powder, granules, beads, spheroids, spheres, microspheres, spherules, beadlets, multiparticulates, microcapsules, millispheres and the like or mixture thereof, irrespective of their size, shape or morphology. The term “matrix tablet” refers to a tablet comprising a “controlled release matrix” or “modified release matrix” as defined herein. A “matrix tablet” may or may not comprise a functional coating.

The term “extended release composition” herein refers to any composition or dosage form that comprises an active drug and which is formulated to provide a longer duration of pharmacological response after administration than is ordinarily experienced after administration of a corresponding immediate release composition. The terms “extended release composition” describe a solid pharmaceutical composition comprising an active agent such as riociguat, and at least one release controlling agent, wherein the release controlling agent is capable of retarding the release of the active agent from the composition, compared with an immediate release composition of the active agent. The term “extended release” as used herein, refers to a release profile to effect delivery of riociguat over an extended period of time, as being between about 1 hour to about 2, 4, 6, 8, 12, 16 or 24 hours.

Extended release compositions include, those compositions described elsewhere as “modified release”, “controlled release”, “delayed release”, “sustained release”, “prolonged release”, “programmed release”, “time release” and/or “rate controlled” compositions or dosage forms. The pharmaceutical composition can optionally have one or more coatings such as film coating, sugar coating, enteric coating, bioadhesive coating and other coatings known in the art. These coatings help pharmaceutical formulations to release the drug at the required site of action.

The terms “release controlling agent” or “release controlling material” or “release controlling polymer” and “agent capable of retarding release” describe an agent present in a solid pharmaceutical composition comprising an active agent such as riociguat, wherein the release controlling agent is capable of retarding the release of the active agent from the composition, compared with an immediate release composition of the active agent.

“Riociguat” is used in broad sense to include not only riociguat per se (free base) but also its pharmaceutically acceptable salts, solvates, esters, hydrates, enantiomers, derivatives, isomers, stereoisomers, diastereomers, metabolites, polymorphs, co-crystal and prodrugs thereof. Polymorph may refer to various crystalline and amorphous forms of riociguat or any mixture thereof in any ratio.

The term “excipient” means one or more pharmacologically inactive components comprising diluents, binders, disintegrants, controlled release polymers/release retarding agents, antioxidants, lubricants, glidants, surfactants, plasticizers, wetting agents, solubilizers, stabilizers, anticaking agents, antifoaming agents, alkaline agents, film-forming polymers, opacifiers, ion-exchange resins and coloring agents or the like. Co-processed excipients are also covered under the scope of the present invention. Further, the excipient may be in the form of powders or in the form of a dispersion. Combinations of excipients performing the same function may be used to achieve desired formulation characteristics.

The term “patient” and/or “subject” are used interchangeably herein. In some embodiments, the patient or subject is a human. In further embodiments, the patient or subject is an animal. In some embodiments, the human can be of any age such as adult, adolescent, paediatric or geriatric. “Adverse effects” or “side-effects” as used herein, means physiological effects to various systems in the body such as digestive system, nervous system, cardiovascular systems, and body as a whole, which cause pain and discomfort or any other unwanted effect to the individual.

As used in this specification, the singular forms “a”, “an”, and “the” include plural references unless the context clearly dictates otherwise. Thus, for example, a reference to “a process” includes one or more processes, and/or steps of the type described herein and/or which will become apparent to those persons skilled in the art upon reading this disclosure.

Unless otherwise stated the weight percentages expressed herein are based on the final weight of the composition or formulation.

Unless the context requires otherwise, throughout the present specification and claims, the word “comprise” and variations thereof, such as, “comprises” and “comprising” are to be construed in an open, inclusive sense, that is as “including, but not limited to”.

The use of the term “or”/“and/or” in the claims is used to mean “and/or” unless explicitly indicated to refer only to alternatives or the alternatives are mutually exclusive, although the disclosure supports a definition that refers to only alternatives and “and/or.”

As used herein, the term “about” means±approximately 20% of the indicated value, such that “about 10 percent” indicates approximately 08 to 12 percent.

As used herein, the term “therapeutic agent” means an agent utilized to treat, combat, ameliorate, prevent or improve an unwanted condition or allergy or disease of a patient.

“Administering” a composition may be accomplished by oral administration and or by other methods alone or in combination with other known techniques.

The term “method for the treatment” or “therapeutically effective amount” is such that when administered, the pharmaceutical composition of present invention provides desired therapeutic effect in treatment of pulmonary hypertension and related diseases as mentioned herein. The dosage administered to a patient can be as single or multiple doses depending upon a variety of factors, including the drug's administered pharmacokinetic properties, the route of administration, patient conditions and characteristics (sex, age, body weight, health, size, etc.), and extent of symptoms, concurrent treatments, frequency of treatment and the effect desired. “Improvement” means e.g. a decreased incidence of side effects of riociguat.

The term “stable” refers to the compositions of present invention, wherein the amount of the active ingredient of a formulation do not deviate from the initial amount by more than the values given in the specification or the guidelines of the common Pharmacopoeias or loss in active ingredient is less than 50% (40%, 30%, 20%, 10%, 5%) of the initial content after being stored for at least 1 month, preferably for at least 2 months, preferably for at least 3 months, more preferably for at least 6 months, more preferably for at least 12 months or more preferably for at least 24 months. The stability of the composition may be evaluated at “long term” conditions 25° C./60% RH, at intermediate condition 30° C./65% RH, at “accelerated conditions” 40° C./75% RH, at refrigerated conditions 2-8° C., in the final container either measured as the loss in content of active ingredient. Stability testing may be conducted according to the current guidelines by ICH and USFDA. The purity of riociguat in compositions as per the present invention ranges from at least 100%, 99.99%, 99%, 98%, 97%, 96% or 95%. The assay of riociguat in compositions as per the present invention ranges from at least 95% to 105% w/w.

The pharmaceutical compositions of the present invention comprise riociguat from about 0.05 μg to about 100 mg. In some embodiments the compositions of the present invention comprise riociguat ranges from 0.05 μg to about 100 mg, from 0.05 μg to about 50 mg, 0.05 μg to about 40 mg, 0.05 μg to about 30 mg, 0.05 μg to about 20 mg, 0.05 μg to about 10 mg, 0.05 μg to about 5 mg, 0.05 μg to about 4 mg, 0.05 μg to about 3 mg, 0.05 μg to about 2 mg, 0.05 μg to about 1.5 mg, 0.05 μg to about 1 mg, 0.05 μg to about 0.5 mg or less. The formulations disclosed herein can deliver a daily therapeutic dose of riociguat for a period of at least 1 day, 2 days, 3 days, 1 week, 2 weeks, 4 weeks, 8 weeks, 12 weeks, 24 weeks or at least 28 weeks. In an embodiment, the composition comprises riociguat in an amount from about 0.5 mg to about 7.5 mg.

The extended release composition as per present invention may also show an increased initial release (“burst”) of riociguat, for example of about 1 to about 25%, of the total amount of riociguat in the composition. Such formulations with an initial burst of composition within the first hour are encompassed by the present invention so long as the dissolution rates at the time points (e.g. at 2, 4 or 8 hours etc.) are within the ranges further disclosed and claimed herein. Such controlled burst may be achieved by applying an immediate release outer coating to an extended release formulation, wherein said immediate release coating comprises a predefined amount of riociguat to be released as burst. Preferably the initial burst of riociguat compared to the preferred dissolution profiles as disclosed herein are below 30%, preferably below 20%, more preferably below 10% and even more preferably below 5% of the total riociguat content of the formulation.

The present invention relates to an extended release pharmaceutical composition suitable for once daily dosing comprising riociguat and at least one or more pharmaceutically acceptable excipients.

The present invention relates to an extended release pharmaceutical composition suitable for twice daily dosing comprising riociguat and at least one or more pharmaceutically acceptable excipients.

The present invention also relates to extended release compositions of riociguat and at least one or more pharmaceutically acceptable excipients and processes for preparing such compositions.

In another embodiment, the present invention relates to extended release compositions comprising riociguat and at least one or more pharmaceutically acceptable excipients selected from the group comprising of diluent, binder, disintegrant, controlled release polymer, antioxidant, lubricant, glidant, surfactant, plasticizer, wetting agent, solubilizer, stabilizer, anticaking agent, antifoaming agent, alkaline agent, film-forming polymer, opacifiers and coloring agent.

In another embodiment, the present invention relates to an extended release solid oral pharmaceutical composition suitable for once or twice daily dosing such as tablet or capsule comprising riociguat and at least one or more pharmaceutically acceptable excipients. In another embodiment, the composition is in the form of granules, tablets, pellets or capsules. In another embodiment, the tablet composition is a matrix tablet or multiparticulate tablet dosage form.

In another embodiment, the present invention relates to an extended release matrix tablet suitable for once or twice daily dosing comprising riociguat and at least one or more pharmaceutically acceptable excipients.

In another embodiment, the present invention relates to an extended release coated tablet dosage form suitable for once or twice daily dosing comprising riociguat and at least one or more pharmaceutically acceptable excipients.

In another embodiment, the present invention relates to a pharmaceutical composition for the oral administration comprising: a) riociguat as active ingredient, and b) at least one or more pharmaceutically acceptable excipients comprising at least one release controlling agent.

In another embodiment, the present invention relates to an extended release pharmaceutical composition suitable for once or twice daily dosing comprising riociguat and at least one or more hydrophilic release controlling material(s).

In another embodiment, the present invention relates to an extended release pharmaceutical composition suitable for once or twice daily dosing comprising riociguat and at least one or more hydrophobic release controlling material(s).

In another embodiment, the present invention relates to a pharmaceutical composition for the oral administration comprising: a) about 0.5 to about 10 mg of riociguat as active ingredient, and b) at least one excipient being a riociguat release controlling agent and being present in the matrix and/or in the coating of said solid composition, and c) at least one or more pharmaceutically acceptable excipients.

In another embodiment, the present invention relates to a pharmaceutical composition comprising: a) an inert core optionally coated with one or more water soluble pharmaceutically acceptable polymers, b) a drug layer comprising riociguat, one or more release controlling polymers and one or more pharmaceutically acceptable excipients, c) optionally a seal coat comprising a water soluble polymer, d) extra granular excipients for dispensing the coated multiparticulates into capsules or compressed into tablets.

In another embodiment, the present invention relates to an extended release multiparticulate pharmaceutical composition comprising: a) an inert core, b) drug layer comprising riociguat, c) optionally a seal coat: comprising one or more water soluble or water insoluble polymers, d) extended release coat comprising a release controlling polymer and at least one or more pharmaceutically acceptable excipient, and e) optionally a seal coat, f) an outer coat comprising one or more pharmaceutical excipients.

Another embodiment of the present invention provides an extended release composition of riociguat comprising riociguat complexed with ion-exchange resin particles to form drug resin particles, which are coated with an extended release coating comprising a release-controlling agent.

Another embodiment of the present invention provides an extended release composition of riociguat comprising riociguat complexed with sodium polystyrene sulfonate resin to form drug resin particles, which are coated with an extended release coating comprising a release-controlling agent.

In another embodiment, hydrophilic release controlling material is selected from the group comprising hydroxypropyl methylcellulose, hydroxypropyl cellulose, povidone, polyethylene glycols, vinyl acetate copolymers, polysaccharides as alginates, xanthan gum, chitosan, carrageenan, dextran, polyalkylene oxides as polyethylene oxide, methacrylic acid copolymers, maleic anhydride/methyl vinyl ether copolymers, carbomers.

In another embodiment, hydrophobic release controlling material is selected from the group comprising ethyl cellulose, cellulose acetate, polyvinyl acetate dispersion, cellulose acetate phthalate, cellulose triacetate, poly (methyl methacrylate), poly (ethyl methacrylate), poly (butyl methacrylate), poly (isobutyl methacrylate), poly (hexyl methacrylate), poly (isodecyl methacrylate), poly (lauryl methacrylate), poly (phenyl methacrylate), poly (methyl acrylate), poly (isopropyl acrylate), poly (isobutyl acrylate), poly (octadecyl acrylate), beeswax, carnauba wax, paraffin wax, cetostearyl alcohol, stearyl alcohol, cetyl alcohol, glyceryl monostearate, glycerol monooleate, acetylated monoglycerides, glyceryl behenate and hydrogenated vegetable oils.

In an embodiment, the amount of riociguat in the composition of the present invention is from about 0.01% to about 95% of the total weight of the composition. In a preferred embodiment, the amount of riociguat is from about 5% to about 95% of the total weight of the composition. In a preferred embodiment, the amount of riociguat is from about 1% to about 50% of the total weight of the composition. In a preferred embodiment, the amount of riociguat is from about 0.01% to about 20% of the total weight of the composition. In a preferred embodiment, the amount of riociguat is from about 0.01% to about 10% of the total weight of the composition. In a preferred embodiment, the amount of riociguat is from about 0.01% to about 5% of the total weight of the composition. In a preferred embodiment, the amount of riociguat is from about 0.01% to about 3% of the total weight of the composition.

In another embodiment, the present invention relates to an extended release tablet dosage form suitable for once or twice daily dosing comprising riociguat and at least one or more pharmaceutically acceptable excipients, wherein the composition is free of any swelling agent selected from polyethylene oxide derivatives and/or, carbopol derivatives. In a preferred embodiment, the composition is free from polyethylene oxide or derivatives.

Glidants are usually added to compositions after granulation and drying of the particles to facilitate the flow of dried granules (extra-granular). Glidants such as talc, colloidal silicon dioxide, are extremely fluffy and bulky, and often require a sieving stage. However, during the sieving process, a substantial amount of airborne particles is produced. These airborne particles are a potential safety hazard, and, thus, require the use of safety equipment and other safety precautions by scientists handling the materials. Further, particles of glidants are very small and thus the total surface area is very large, relative to the volume. This small size and large surface area are the possible cause of instability due to the direct interaction between the glidant particles and the active ingredient. Thus, with an objective to reduce hazards to workers and improve the stability of riociguat compositions there is an unmet need for glidant free riociguat formulations and methods of preparing such formulations. The present invention provides such formulations and methods.

In another embodiment, the present invention relates to, an extended release tablet dosage form suitable for once or twice daily dosing comprising riociguat and at least one or more pharmaceutically acceptable excipients, wherein the composition is free of glidant. In a preferred embodiment, the excluded glidant is talc. In a preferred embodiment, the excluded glidant is colloidal silicon dioxide.

In another embodiment, the present invention is directed to an extended release composition comprising: a) from about 0.01% to about 50% of riociguat as active pharmaceutical ingredient and b) about 1% to about 99% of at least one or more pharmaceutically acceptable excipients.

In another embodiment, the present invention is directed to an extended release tablet suitable for once or twice daily dosing comprising: a) about 0.01% w/w to about 50% w/w of riociguat, b) about 0.1% to about 80% w/w of one or more release controlling materials, c) at least one or more other pharmaceutically acceptable excipients. In another embodiment, the release controlling materials are present from about 0.1% w/w to about 40% w/w of the composition.

In another embodiment, the present invention also relates to, a solid oral pharmaceutical composition, preferably for the once or twice daily administration comprising: a) about 0.5 to about 10 mg of riociguat as active ingredient (preferably representing about 0.01% to about 50 wt % of the total weight of the composition), and b) at least one excipient being a riociguat release controlling agent being present in an amount of 0.1% to 50 wt % relative to the total weight of the composition and/or being a coating of said solid composition in an amount of about 5% to about 50 wt % relative to the total weight of the formulation, and c) at least one or more pharmaceutically acceptable excipients.

In another embodiment, the present invention relates to a solid oral pharmaceutical of riociguat, preferably for the once or twice daily administration comprising: a) about 0.5 to about 10 mg of riociguat as active ingredient (preferably representing about 0.01% to about 60 wt % of the total weight of the composition), and b) at least one excipient being a riociguat release controlling agent being present in an amount of 5% to 50 wt % relative to the total weight of the composition and/or being a coating of said solid composition in an amount of about 5% to about 50 wt % relative to the total weight of the formulation, and c) at least one or more pharmaceutically acceptable excipients which may optionally comprise one or more of the group comprising diluents, binders, disintegrants, lubricants in a total amount of between about 5% and 80 wt % relative to the total weight of the formulation.

In another embodiment, the present invention relates to a solid oral pharmaceutical composition comprising: a) riociguat in an amount of about 0.01% to 50 wt %, b) a matrix retarding agent in an amount of about 5% to 40 wt %, c) a diluent in an amount of 0 to about 85 wt %, d) a binder in an amount of 0 to about 20 wt %, e) a disintegrant in an amount of 0 to about 30 wt %, f) a lubricant and a glidant in an amount of 0 to 25%, and optionally, g) a non-functional film coat preferably in an amount of 0 to 10 wt %, wherein all amounts relative to the total weight of the composition.

In another embodiment, the present invention relates to, a solid oral pharmaceutical composition comprising: a) about 0.01% to about 50% by weight of riociguat, b) about 5% to about 85% by weight of a diluent, c) 0 to about 30% by weight of a binder, d) 0 to about 30% by weight of a disintegrant, e) 0 to about 40% by weight of a release controlling polymer, f) 0 to about 5% by weight of a cushioning agent, g) 0 to about 10% by weight of a surfactant, h) about 0.1% to about 10% by weight of a lubricant, and i) 0 to about 5% by weight of a glidant.

In another embodiment, the present invention relates to an extended release pharmaceutical composition suitable for once or twice daily dosing comprising: a) riociguat in an amount of about 0.01% to about 10% by weight, b) a release controlling material selected from the group consisting of hydrophilic release controlling material or hydrophobic release controlling material or combination thereof in an amount of about 25% to about 35% by weight, and c) one or more other pharmaceutically acceptable excipients, wherein the composition is free of glidant and the ratio of riociguat to release controlling material in the composition is more than 1:15. In other embodiment, one or more other pharmaceutically acceptable excipients are selected from the group consisting of diluent, binder, disintegrant, antioxidant, surfactant, plasticizer, stabilizer, anticaking agent, anti-foaming agent, lubricant, film-forming polymer, opacifier and coloring agent.

In another embodiment, the present invention relates to an extended release solid oral pharmaceutical composition comprising: a) riociguat in an amount of about 0.01% to about 10% by weight, b) a matrix retarding agent selected from the group consisting of hydroxypropyl methylcellulose, ethyl cellulose, or combination thereof in an amount of about 25% to about 35% by weight, c) a diluent selected from the group consisting of lactose, microcrystalline cellulose, mannitol, starch or combination thereof in an amount of about 30% to about 80% by weight, d) a binder selected from the group consisting of povidone, hydroxypropyl methylcellulose, hydroxypropyl cellulose or combination thereof in an amount of about 0.1% to about 10% by weight, and e) a lubricant selected from the group consisting of magnesium stearate, stearic acid, sodium stearyl fumarate or combination thereof in an amount of about 0.01% to about 3% by weight. In another embodiment, matrix retarding agent is present in an amount of less than 35% by weight.

In another embodiment, the present invention relates to an extended release pharmaceutical tablet composition comprising: a) riociguat in an amount of about 0.01% to about 10% by weight, b) a release controlling material selected from the group consisting of hydroxypropyl methylcellulose, hydroxypropyl cellulose, methacrylic acid copolymers, ethyl cellulose, cellulose acetate or combination thereof in an amount of about 25% to about 35% by weight, c) a diluent selected from a group consisting of lactose, cellulose, microcrystalline cellulose, mannitol, starch, pregelatinized starch, or combination thereof in an amount of about 30% to about 80% by weight; and d) a binder selected from a group consisting of polyvinylpyrrolidone, hydroxypropyl methylcellulose, hydroxypropyl cellulose, or combination thereof in an amount of about 0.1% to about 10% by weight, wherein the composition is free of any glidant and the ratio of riociguat to release controlling material in the composition is more than 1:15. In another embodiment, release controlling material is present in an amount of less than 35% by weight.

In a preferred embodiment, the formulation is free of any glidant such as colloidal silicon dioxide and talc. In a preferred embodiment, the formulation is free of swelling agents. In a preferred embodiment, the formulation is free of polyethylene oxide. In a preferred embodiment, the ratio of riociguat to release controlling material in the composition is at least 1:20. In a preferred embodiment, the ratio of riociguat to diluent in the composition is at least 1:30. In a preferred embodiment, the ratio of riociguat to diluent in the composition is at least 1:40. In a preferred embodiment, the ratio of riociguat:release controlling material:diluent in the composition is at least 1:20:40.

In another embodiment, the present invention relates to, a glidant free extended release pharmaceutical tablet composition comprising: a) riociguat in an amount of about 0.01% to about 10% by weight, b) hydroxypropyl methylcellulose and/or ethyl cellulose in an amount of about 25% to about 35% by weight, c) microcrystalline cellulose in an amount of at least about 25% by weight, d) polyvinylpyrrolidone in an amount of at least about 1% by weight; and e) magnesium stearate in an amount of at least about 0.1% by weight, wherein the composition exhibits at least 10% drug release in 4 hours, at least 15% drug release in 6 hours, at least 20% drug release in 8 hours, and at least 40% drug release in 12 hours when measured in 900 ml of 6.8 phosphate buffer and 0.1% sodium lauryl sulfate (SLS) using a USP II apparatus (Paddle) at a temperature of 37±0.5° C. and a rotation speed of 75 to 100 revolutions per minute.

In another embodiment, the present invention relates to an extended release solid oral pharmaceutical composition of riociguat comprising:
i. an inert inner core,
ii. optionally a water-soluble seal or seal coating layer consisting essentially of water-soluble polymers,
iii. a first coating on top of the inner core comprised of riociguat in an amount of about 0.01% to about 10% by weight and a binder selected from the group consisting of povidone, hydroxypropyl methylcellulose, hydroxypropyl cellulose, polyvinyl alcohol, starch, methylcellulose, ethylcellulose, carboxymethyl cellulose, sucrose solution, dextrose solution in an amount of about 0.1% to about 20% by weight,
iv. optionally a water-soluble seal or seal coating layer consisting essentially of water-soluble polymers,
v. an extended coating layer comprises of at least one water-insoluble cellulose derivative in an amount of about 5% or less by weight, and
vi. an extra-granular part comprises of:
   a) a diluent selected from the group consisting of lactose, microcrystalline cellulose, mannitol, starch or combination thereof in an amount of about 10% to about 90% by weight,
   b) a disintegrant selected from the group consisting of crospovidone, croscarmellose sodium, carboxymethyl cellulose sodium in an amount of about 0.1% to about 20% by weight, and
   c) a lubricant selected from the group consisting of magnesium stearate, stearic acid, sodium stearyl fumarate in an amount of about 0.01% to about 3% by weight.

In a preferred embodiment, the inner core is an inert non-pareil sugar sphere. In a preferred embodiment, the core is devoid of any cellulose-based excipients. In a preferred embodiment, the core is devoid of microcrystalline cellulose. In a preferred embodiment, the drug and binder is present in a ratio less than 1:3 or less than 1:2. Preferably the drug and binder is in a ratio of 1:1. In a preferred embodiment, the controlled release polymer has a viscosity of less than 10 cps. In a preferred embodiment, the controlled release polymer has a viscosity of less than 10 cps. Preferably, the controlled release polymer is ethyl cellulose having a viscosity of 7 cps. In a preferred embodiment, the controlled release polymer is used in about 20% weight gain or less, about 10% weight gain or less by the total weight of the core or drug layered composition. In a preferred embodiment, the controlled release polymer is used in about 5% weight gain or less by the total weight of core or drug layered composition. In a preferred embodiment, the composition is free of any glidant. In a preferred embodiment, the multi-particulate delivery system contains particles having a mean diameter of about 400 to about 1200 microns.

In another embodiment, the oral pharmaceutical composition is in the form of a tablet or capsule, wherein the ratio of riociguat to a release controlling polymer in the composition ranges from about 1:1 to about 1:50. In particular, the ratio of riociguat to release modifying polymer in the composition is about 1:1 to about 1:40.

More particular, the ratio of riociguat to release modifying polymer in the composition is about 1:1 to about 1:20. In another embodiment, the ratio of riociguat to binder in the composition ranges from about 0.5:1 to about 1:50. In another embodiment, the ratio of riociguat to binder in the composition is at least 1:5 or less. In another embodiment, the ratio of riociguat to binder in the composition is at least 1:1 or more. In another embodiment, the ratio of riociguat to binder in the composition is at least 1:1 or less. In another embodiment, the ratio of riociguat to binder in the composition is 1:1. In another embodiment, the ratio of riociguat to binder in the composition is less than 1:2.

In another embodiment of the invention, the pharmaceutical composition is prepared by wet granulation (rapid mixture granulation, fluid bed granulation, spray drying), dry granulation, dry blending, dry mixing and direct compression. Other formulation techniques are also contemplated within the scope of the present invention such as extrusion-spheronization, hot melt extrusion, melt-granulation, freeze-drying, spray drying, mass extrusion and molding, solid dispersion (solvent evaporation, melting method, kneading method, co-grinding method, co-precipitation method, freeze-drying, spray-drying, coating, or adsorbing the drug onto carrier particles), multi-particulate based technology-pelletization and drug-ion exchange resin complexation.

In an embodiment, the extended release compositions may be prepared by mixing riociguat with one or more rate-controlling polymers along with one or more pharmaceutically acceptable excipients to form a blend. The blend can be lubricated and formulated into suitable dosage form.

In an embodiment, the extended release compositions may be prepared by mixing and granulating riociguat with one or more rate-controlling polymers along with one or more pharmaceutically acceptable excipients to form granules. The granules can be dried. The dried granules can be milled, mixed with other pharmaceutically acceptable excipients, lubricated and formulated into suitable dosage form. Further, the dosage form can be functionally coated with one or more rate-controlling polymers.

In another embodiment of the invention, there is provided a process of preparing a riociguat composition, includes the steps of: a) mixing suitable amounts of riociguat, a matrix retardation agent, and optionally a binder, preferably in an aqueous solvent, b) granulating the mixture produced in step a), preferably by wet granulation, c) adding the remaining matrix excipients and mixing with the granules produced in step b), pressing the blend produced in step c) to tablets, and e) optionally applying a coating to the tablets obtained in step d).

In another embodiment of the invention, the active ingredient in wet granulation process can be placed within the internal phase as a solid material, or in the external phase as a solid material, or be dissolved in the liquid phase or a mixture thereof.

In another embodiment of the invention, there is provided a process of preparing riociguat composition, includes the steps of: a) granulating riociguat, a release controlling agent and diluent using a binder solution, b) drying the granules of step a), c) lubricating the dried granules of step b), and d) compressing the lubricated granules of step c) using punching machine or filing them into capsules.

Another embodiment of the present invention also provides a process for the preparation of a solid oral multiparticulate pharmaceutical composition of riociguat comprising the steps of; (a) dry blending the drug and excipients, (b) wet granulation (aqueous or non-aqueous) of the mass, (c) extrusion through a screen of defined mesh size to compact the wet mass into cylindrical strands, (d) spheronization of cylindrical strands in a spheronizer to convert the cylindrical strands into spheres, (e) seal coating of the spheres, (f) coating of the spheres with release controlling polymers over the seal coating, and (g) optionally coated with an outer seal coat over the extended release coating layer and finally applying outer coat one or more pharmaceutically acceptable excipients.

In a further embodiment, the process of preparing extended release compositions comprising multiple-unit particles, which process comprises steps of: a) coating inert core with riociguat with one or more rate-controlling polymers to form drug coated pellets, b) optionally providing one or more layers of non-functional coating or functional coating of rate-controlling polymer over the drug coated pellets, c) filling the pellets prepared in step b) in hard gelatin capsules or compressing into tablets.

In another embodiment of the present invention relates to a process for the preparation of a multiparticulate pharmaceutical composition, wherein the process comprises: a) coating the inert core with a drug layer comprising riociguat b) optionally applying the seal coating of a water soluble/insoluble polymer on the drug coated core, c) coating the resultant product with extended release coat comprising a release controlling polymer and at least one or more pharmaceutically acceptable excipient, d) optionally applying a seal coat on the extended release coated core and e) applying outer coat on the core of step d) wherein outer core comprises of one or more pharmaceutical excipients.

In one embodiment, the extended release composition is designed in such a way so that it may be administered in a minimum number of daily doses from which the drug is uniformly released over a desired extended period of time.

In another embodiment, the extended release composition exhibits desired formulation technical attributes such as particle size, bulk density, tapped density, ease of manufacturing, assay, drug release, dosage regimen, stability, patient compliance and commercially viable and other requirements also.

In another embodiment, the extended release composition as per present invention exhibits a dissolution profile which is suitable for once or twice a day dosage regimen.

In another embodiment, the extended release composition as per present invention provides a continuous and stable drug release, effective blood drug level, better therapeutic effects and low side-effects, and thus overcome the challenges of conventional immediate release oral dosage forms by providing good therapeutic efficacy.

In one embodiment, the present invention provides a composition comprising riociguat and at least one or more pharmaceutically acceptable excipients having improved systemic absorption. In one embodiment, the present invention provides an extended release composition which will provide the therapeutic amount of drug for long duration of time by controlling the release rate to achieve the desired pharmacologic effect and improved systemic absorption. The inventors of present invention considering the physicochemical properties of riociguat to provide an extended release composition comprising riociguat with one or more pharmaceutically acceptable excipients, wherein the composition is able to achieve the desired pharmacokinetic profile for longer duration of time.

In one embodiment, the granules of the present invention may have a mean diameter of from about 10 μm to about 2000 μm or about 200 μm to about 1000 μm (D50). In one embodiment, the pellets of the present invention may have a mean diameter of from about 10 μm to about 3000 μm or from about 200 μm to about 2000 μm (D50). In another embodiment, the multiparticulates of the present invention have an average diameter of about 0.5 mm to about 5.0 mm.

In another embodiment the composition of riociguat includes particle size of riociguat, having a particle size distribution such that D90 is less than about 200 μm, D50 is less than about 100 μm and D10 is less than about 50 μm. Preferably, particle size distribution is D90 is less than about 100 μm, D50 is less than about 70 μm and D10 is less than about 30 μm. Preferably, D90 of the particles is less than 100 μm, 60 μm, 50 μm, less than 40 μm, less than 35 μm, or less than 25 μm, D50 of the particles is less than 40 μm, less than 35 μm, less than 30 μm, or less than 25 μm and D10 of the particles is less than 20 μm, less than 17 μm, less than 15 μm, or less than 12 μm. The terms D10, D50, and D90 used herein as per the present invention indicate that 10%, 50% and 90% of the distribution is below this value. The particle size can be achieved by any well-known particle size reduction processes, such as sifting, milling, micronization, fluid energy milling, media milling, ball milling, milled through high pressure homogenizer, air jet milling, and the like. The particle size can be measured by suitable techniques such as Laser light scattering (e.g. Malvern Light Scattering), Coulter counter, microscopy and any other technique known in the art.

In another embodiment, the composition of riociguat is stable for at least 3 months, preferably for 6 months, more preferably for 12 months and more preferably for 24 months when stored at room temperature.

In another embodiment, the riociguat is homogenously dispersed throughout the pharmaceutical composition. The phrase “homogenous” as used herein, means that the active ingredients are substantially evenly dispersed throughout the part of the finished dosage form which comprises them. The homogeneity of active ingredients in a tablet may be ascertained by means of the drug uniformity measure.

In another embodiment, the present invention provides an extended release pharmaceutical composition suitable for once or twice daily dosing comprising riociguat and at least one or more pharmaceutically acceptable excipients so that upon oral administration the maximum concentrations (Cₘₐₓ) of riociguat in plasma are statistically significantly lower than the immediate release formulation given thrice daily, and area under the plasma concentration-time curve (AUC) and the minimum plasma concentration are maintained over 24 hours.

In further embodiment of the invention, the extended release compositions as per present invention is bioequivalent to the marketed conventional immediate release composition of riociguat (Adempas®) administered thrice daily.

In further embodiment of the invention, the compositions as per present invention exhibit no significant difference in both rate and extent of absorption of riociguat as compared to marketed composition under trade name ADEMPAS®. In further embodiment of the invention, “Bioequivalency” may be is determined by a 90% Confidence Interval (CI) of between 0.80 and 1.25 for both Cₘₐₓ and AUC under USFDA regulatory guidelines, or a 90% CI for AUC of between 0.80 to 1.25 and a 90% CI for Cₘₐₓ of between 0.70 to 1.43 under the European regulatory guidelines (EMEA). The bioequivalence studies can be carried out between ADEMPAS® (reference) and compositions of the invention (test) in fasted and/or fed state. The study can be monitored in terms of Cₘₐₓ, AUC, Tₘₐₓ achieved with the test product and the reference product.

In one embodiment, the present invention provides a method of administering an extended release composition as described in the present invention.

In another embodiment of the invention, there is provided a use of therapeutic effective amount of extended release composition of riociguat in the manufacture of medicament for treating hypertension, pulmonary hypertension, pulmonary arterial hypertension, chronic thromboembolic pulmonary hypertension, heart failure, arrhythmia, high blood pressure, angina pectoris, myocardial infarction, stroke, transient ischaemic attacks, arteriosclerosis, erectile dysfunction, osteoporosis, alzheimer's disease, parkinson's disease, multiple sclerosis, depression, schizophrenia, bipolar disorder and migraine.

In one embodiment, the present invention provides riociguat compositions having an improved (i.e. decreased) side effect/efficacy ratio and benefit/risk ratio, and a need for methods of using such formulations, such formulations and methods thereby offering the option for treatment regimens with increased doses of riociguat. These improved formulations containing increased doses of riociguat could be administered once or twice daily, and would offer an additional advantage for patients compared to the presently available immediate release formulations for three times daily use.

In an embodiment the composition as per present invention is packed in a suitable container selected from a foil, a pouch, a sachet, capsule, bottle, blister, container or other suitable packages including calendar packs.

Any pharmaceutically acceptable granulating agent can be used for wet granulation. Preferable granulating solvents include, but are not limited to, aqueous, non-aqueous, alcoholic, hydro-alcoholic, ether, acidic solvents, water, esters such as ethyl acetate, ketones such as acetone, alcohols such as methanol, ethanol (including ethanol 95%), isopropanol, butanol, dichloromethane, chloroform, dimethyl acetamide, dimethyl sulfoxide, ether (such as tetrahydrofuran, methyl tert-butyl ether, 1,4-dioxane and combinations thereof), diethyl ether, hydrochloric acid (such as 1N hydrochloric acid), mixtures of one or more alcohols and water and any combinations or mixtures thereof. In a further embodiment, solvents used as per present invention are mixtures of one or more of alcohol and water, in various volume ratios. In some embodiment, suitable organic or inorganic acids selected from the group comprising citric acid, malic acid, fumaric acid, tartaric acid and hydrochloric acid can also be added in granulation process or in granulation solution.

Various useful fillers or diluents include, but are not limited to, microcrystalline cellulose (“MCC”), sodium alginate, silicified MCC, microfine cellulose, lactitol, cellulose acetate, kaolin, glucose, lactose, maltose, fructose, sucrose, trehalose, starch, pregelatinized starch (PGS), sugars, sugar alcohols such as mannitol, D-mannitol, xylitol, maltitol, sorbitol, dextrates, dextrin, maltodextrin, compressible sugar, confectioner's sugar, dextrose, polydextrose, simethicone, calcium carbonate, magnesium carbonate, calcium sulfate, magnesium aluminium silicate, calcium phosphate, dibasic calcium phosphate, tribasic calcium phosphate, magnesium carbonate, magnesium oxide, isomalt, chitosan, sodium chloride, magnesium aluminometasilicate, fats, waxes, fatty alcohols or fatty acid esters, mineral oils, vegetable oils, mixture of microcrystalline cellulose and mannitol or any mixtures thereof. The amount of diluent according to the present invention ranges from about 0 to about 90% by weight of composition. In an embodiment, the diluent according to the present invention is present in an amount of about 90% or less, 80% or less, e.g. 70% or less, 60% or less, 50% or less, 40% or less, 30% or less, 20% or less.

Various useful binders include, but are not limited to, acacia, guar gum, xanthan gum, alginic acid, sodium alginate, alginate salts (e.g. potassium, calcium), dextrin, carbomer, maltodextrin, methylcellulose, ethyl cellulose, hydroxyethyl cellulose, hydroxypropyl cellulose (HPC), hydroxypropyl methylcelulose (HPMC), hydroxyethylmethyl cellulose, carboxymethyl cellulose sodium, cottonseed oil, povidone (PVP), ceratonia, dextrose, polydextrose, starch, modified starch, pregelatinized starch, hydroxypropyl starch, sodium carboxymethylated starch, gelatin, hydrogenated vegetable oil type I, lactose, microcrystalline cellulose, silicified microcrystalline cellulose, polyethylene oxide, dibasic calcium phosphate derivatives, magnesium carbonates, magnesium aluminium silicate, sodium bicarbonate, polyethylene glycol, copovidone, polyvinyl acetate, polyvinyl alcohol, poloxamers, ethylcellulose, hydroxyethylcellulose, hydroxypropylmethylcellulose acetate phthalate, low substituted hydroxypropyl cellulose, hydroxypropylmethylcellulose acetate succinate, shellac, acrylic resins, materials derived from protein, polymethacrylates, methacrylic acid copolymer type A, methacrylic acid copolymer type B, methacrylic acid copolymer type C, ammonio methacrylate copolymer type A, ammonio methacrylate copolymer type B, basic butylated methacrylate copolymer, sodium hyaluronate, dextrate, carrageenan, chitosan, pectin, cethyl palmitate, glyceryl behenate, glyceryl monostearate, glyceryl palmitostearate, monoglycerides, diglycerides, triglycerides, glycerine esters, fatty alcohols, and fatty acid esters and any mixtures thereof. Binder can be present in powder form or as a dispersion or mixture of both, in intra and/or extra granular part of the composition. The amount of binder according to the present invention ranges from about 0 to about 50% by weight of composition. In an embodiment, the binder according to the present invention is present in an amount of about 50% or less, 40% or less, e.g. 30% or less, 20% or less, 10% or less, 5% or less, 3% or less.

Various useful disintegrants and/or super-disintegrants include, but are not limited to, croscarmellose sodium, carboxymethyl cellulose sodium, carboxymethyl cellulose calcium, povidone, crospovidone, polacrilin potassium, sodium starch glycolate, alginic acid, sodium alginate, calcium phosphate, colloidal silicon dioxide, docusate sodium, guar gum, low substituted hydroxypropyl cellulose, magnesium aluminum silicate, methylcellulose, microcrystalline cellulose, silicified microcrystalline cellulose, starch, pre-gelatinized starch and/or combinations thereof. The disintegrant can be present in intra-granular or extra-granular part or in both (intra and extra granular) part of the composition. The amount of disintegrant according to the present invention ranges from about 0 to about 40% by weight of composition. In an embodiment, the disintegrant according to the present invention is present in an amount of about 40% or less, 30% or less, e.g. 20% or less, 10% or less, 5% or less.

Various useful surfactants or wetting agents include, but are not limited to, sodium lauryl sulphate, polysorbate (e.g. polysorbate 20, polysorbate 40, polysorbate 60, and polysorbate 80), cetrimide, cetyl alcohol, stearyl alcohol, cetyl stearyl alcohol, cholesterol, polyethylene glycols, polyglycerin fatty acid esters such as decaglyceryl monolaurate and decaglyceryl monomyristate, sorbitan fatty acid esters such as sorbitan monostearate, polyoxyethylene sorbitan fatty acid ester such as polyoxyethylene sorbitan monooleate, polyoxyethylene alkyl ether such as polyoxyethylene lauryl ether, polyoxyethylene castor oil, polyoxyethylenepolyoxy propylene block copolymers such as poloxamer (such as poloxamer 124, poloxamer 188, poloxamer 237, poloxamer 338, and poloxamer 407) and combinations thereof. The amount of surfactant according to the present invention ranges from about 0 to about 30% by weight of composition. In an embodiment, the surfactant according to the present invention is present in an amount of about 30% or less, e.g. 20% or less, 10% or less, 5% or less, or 2% or less or 1% or less.

Suitable glidants include, but are not limited to, calcium silicate, magnesium silicate, magnesium trisilicate, stearic acid and its derivatives or esters like magnesium stearate, calcium stearate and sodium stearate and the corresponding esters such as sodium stearyl fumarate, talc, colloidal silicon dioxide, tribasic calcium phosphate, starch, starch derivatives or mixtures thereof. The amount of glidant according to the present invention ranges from about 0 to about 20% by weight of composition. In an embodiment, the glidant according to the present invention is present in an amount of about 20% or less, e.g. 10% or less, 5% or less, 3% or less or 2% or less or 1% or less.

Pharmaceutically acceptable lubricants include, but are not limited to, stearic acid, zinc stearate, sucrose stearate, sodium benzoate, hydrogenated vegetable oil, calcium stearate, adipic acid, glyceryl palmitostearate, glycerine monostearate, medium-chain triglycerides, glyceryl behenate, sodium lauryl sulphate, sodium stearyl fumarate, magnesium lauryl sulphate, magnesium stearate and polyethylene glycol. The amount of lubricant according to the present invention ranges from about 0 to about 20% by weight of composition. In an embodiment, the lubricant according to the present invention is present in an amount of about 20% or less, e.g. 10% or less, 5% or less or 3% or less or 2% or less or 1% or less.

Suitable plasticizers include, but are not limited to, triethyl citrate, triacetin, glycerol, polyethylene glycol, lecithin, dibutyl phthalate, dibutyl sebacate, and diethyl phthalate. In an embodiment, the plasticizer according to the present invention is present in an amount of about 20% or less, e.g. 10% or less, 5% or less or 3% or less or 2% or less or 1% or less.

Suitable antioxidants include, but are not limited to, butylated hydroxyl anisole, butylated hydroxyl toluene, tocopherol, ascorbyl palmitate, ascorbic acid, sodium metabisulfite, sodium sulfite, sodium thiosulfate, propyl gallate, and mixtures thereof. In an embodiment, the antioxidant according to the present invention is present in an amount of about 20% or less, e.g. 10% or less, 5% or less or 3% or less or 2% or less or 1% or less.

Suitable alkaline agents include, but are not limited to, pharmacologically alkali metal, alkaline earth metal or metal salts of weak acids such as sodium carbonate which can be anhydrous or hydrous, calcium carbonate and magnesium carbonate and the pharmacologically hydroxides and oxides of alkaline earth and earth metals such as magnesium hydroxide and magnesium oxide. The alkaline agent may constitute from 0 to about 20% by weight of the pharmaceutical composition. In an embodiment, the alkaline agent according to the present invention is present in an amount of about 20% or less, e.g. 10% or less, 5% or less or 3% or less or 2% or less or 1% or less.

Various water-soluble polymers used to form a barrier or seal or film over the core. Examples include but are not limited to cellulose derivatives such as soluble alkyl- or hydroalkylcellulose derivatives such as methylcellulose, hydroxymethyl cellulose, hydroxyethyl cellulose, hydroxypropyl cellulose, hydroxymethylethyl cellulose, hydroxypropyl methylcellulose, vinylpyrrolidone vinyl acetate copolymer (PVP/VA) polymers, sodium carboxymethyl cellulose, polyvinyl alcohol, polyvinyl acetate, polyvinylpyrrolidone, chitosan and derivatives thereof, shellac and derivatives thereof, waxes and fat substances. The water-soluble polymers may be present in an amount of about 0.1% w/w to about 80% w/w. In an embodiment, the water-soluble polymer according to the present invention is present in an amount of about 30% or less, e.g. 20% or less, 10% or less, 5% or less or 3% or less or 2% or less or 1% or less.

Suitable cushioning agents include, but are not limited to, glyceryl monostearate, microcrystalline cellulose, silicon dioxide, silicified microcrystalline cellulose, stearates like calcium stearate and magnesium stearate, lactose, polyethylene glycols, polypropylene glycols and mixtures thereof. In an embodiment, the cushioning agents according to the present invention is present in an amount of about 30% or less, e.g. 20% or less, 10% or less, 5% or less or 3% or less or 2% or less or 1% or less.

In another embodiment of the present invention, release controlling polymers are selected from hydrogel forming polymers, hydrophilic polymers, hydrophobic polymers, gums or any combination or mixture thereof.

Suitable hydrogel-forming polymers include, but are not limited to, polyethylene oxide, hypromellose, hydroxypropyl cellulose, carboxymethyl cellulose sodium, hydroxyethyl cellulose, carboxyvinyl polymers and combinations thereof. Examples of polyethylene oxide (also referred to as PEO) include product names, Polyox WSR-N-60K, PEO-27, PEO-18Z, PEO-15Z, PEO-8Z, Polyox WSR-N-12K, Polyox WSR1105, Polyox WSR-308, Polyox WSR-303, Polyox WSR-N-750, Polyox WSR-N-80, PEO WSR 303, Polyox WSR Coagulant, Polyox WSR-301, Polyox WSR-205, and Polyox WSR-N-10, PEO-4, PEO-3Z, PEO-2, PEO-1Z, PEO-1NF, 3NF, 8NF, 15NF, 18NF, 27NF. The said polymer may be present in an amount of about 0.1% w/w to about 80% w/w. In an embodiment, the polymer according to the present invention is present in an amount of about 60% or less, e.g. 50% or less, 40% or less, 30% or less, 20% or less, 10% or less, 5% or less or 3% or less or 2% or less or 1% or less.

Various useful hydrophilic release controlling materials include, but are not limited to, hydroxypropyl cellulose, povidone, methylcellulose, polyethylene glycols, hydroxypropyl methylcellulose (hypromellose), vinyl acetate copolymers, alginates, xanthan gum, locust bean gum, carrageenan, chitosan, dextran and the like, polyalkylene oxides such as polyethylene oxide and the likes, methacrylic acid copolymers, polyacrylate dispersion 30%, maleic anhydride/methyl vinyl ether copolymers, carbomers and the like. Polymers used in the formulation are one or more grades having a nominal viscosity of about 3 cps to 200,000 cps. The hydrophilic polymer may be present in an amount of about 0.1% w/w to about 80% w/w. In an embodiment, the hydrophilic polymer according to the present invention is present in an amount of about 60% or less, e.g. 50% or less, 40% or less, 30% or less, 20% or less, 10% or less, 5% or less or 3% or less or 2% or less or 1% or less.

Various useful hydrophobic release controlling materials include, but are not limited to, cellulose ether such as ethyl cellulose, polyvinyl acetate, polyvinyl acetate phthalate methylcellulose acetate phthalate (MCAP), methacrylic acid copolymers or its derivatives, methacrylic acid esters neutral polymer, polyvinyl alcohol-maleic anhydride copolymers, hydroxypropyl methyl cellulose phthalate, hydroxypropylmethylcellulose acetate succinate (HPMCAS), hydroxypropylcellulose acetate phthalate (HPCAP), ethylcellulose (EC), cellulose acetate, cellulose propionate (lower, medium or higher molecular weight), cellulose acetate propionate, cellulose acetate butyrate, cellulose acetate phthalate, cellulose triacetate, cellulose acetate trimellitate (CAT), polyacrylate dispersion 30%, poly (methyl methacrylate), poly (ethyl methacrylate), poly (butyl methacrylate), poly (isobutyl methacrylate), and poly (hexyl methacrylate), poly (isodecyl methacrylate), poly (lauryl methacrylate), poly (phenyl methacrylate), poly (methyl acrylate), poly (isopropyl acrylate), poly (isobutyl acrylate), poly (octadecyl acrylate), waxes such as beeswax, carnauba wax, paraffin wax, microcrystalline wax, ozokerite, fatty alcohols such as cetostearyl alcohol, stearyl alcohol, cetyl alcohol, myristyl alcohol, and fatty acid esters such as glyceryl monostearate, glycerol monooleate, acetylated monoglycerides, tristearin, tripalmitin, cetyl esters wax, glyceryl palmitostearate, glyceryl behenate, zein and hydrogenated vegetable oils or their mixtures thereof. Various available grades of EUDRAGIT® including but are not limited to, EUDRAGIT® EPO, EUDRAGIT® RSPO, EUDRAGIT® RL12.5, EUDRAGIT® RLPO, EUDRAGIT® RL30D, EUDRAGIT® S 100, EUDRAGIT® E100, EUDRAGIT® E 12.5, EUDRAGIT® FS 30 D, EUDRAGIT® L 12.5. Combination of two or more Eudragit grades can be used to control the drug release in the present invention. Even the commercially available dispersion of film formers namely, SURELEASE® E-7, EUDRAGIT® NE 30D, AQUACOAT® ECD-30, EUDRAGIT® L-30D, EUDRAGIT® RS 30D, EUDRAGIT® RL 30D may be used for this purpose. Polymers used in the formulation is one or more grades having a nominal viscosity of about 3 cps to 200,000 cps. The insoluble polymers useful in the invention are pharmaceutically acceptable polymers, such as, for example, water insoluble cellulosic derivatives, polyvinyl chloride, amino alkyl methacrylates and the like. Suitable water insoluble cellulose derivatives include polymers such as, for example, ethyl cellulose having a viscosity grade of 7 cps, ethyl cellulose having a viscosity grade of 10 cps, ethyl cellulose having a viscosity grade of 20 cps, ethyl cellulose having a viscosity grade of 100 cps. Preferably, the ethyl cellulose has a viscosity grade of less than 10 cps. More preferably, the ethyl cellulose has a viscosity grade of less than 7 cps. Commercially available examples of ethyl cellulose compositions include products such as, for example, Surelease™, Aquacoat, Ethocel 7 cps, Ethocel 10 cps, Ethocel 20 cps and Ethocel 100 cps from Dow, Ltd. USA. The hydrophobic polymer may be present in an amount of about 0.1% w/w to about 80% w/w. In an embodiment, the hydrophobic polymer according to the present invention is present in an amount of about 70% or less, e.g. 50% or less, 40% or less, 30% or less, 20% or less, 10% or less, 5% or less or 3% or less or 2% or less or 1% or less.

The ion-exchange resins useful in the extended release compositions of the present invention comprise of a pharmacologically inert organic or inorganic matrix containing covalently bound functional groups that are ionic or capable of being ionized under the appropriate conditions of pH. The organic matrix may be synthetic (e.g., polymers or copolymers of acrylic acid, methacrylic acid, sulfonated styrene, sulfonated divinylbenzene), or partially synthetic (e.g., modified cellulose and dextrans). The inorganic matrix preferably comprises silica gel modified by the addition of ionic groups. Covalently bound ionic groups may be strongly acidic (e.g., sulfonic acid, phosphoric acid), weakly acidic (e.g., carboxylic acid), strongly basic (e.g., primary amine), weakly basic (e.g. quaternary ammonium), or a combination of acidic and basic groups. Other types of charged groups can also be used, including any organic group that bears an acidic or a basic functional group, for example, an amine, imine, imidazoyl, guanidine, pyridinyl, quaternary ammonium, or other basic group, or a carboxylic, phosphoric, phenolic, sulfuric, sulfonic or other acidic group. Suitable ion exchange resins are also sold under the trade names Amberlite, Amberlyst and Dowex. Weak cation acid ion exchange resins such as Indion 204, Indion 214, Indion 224, polacrillin potassium as well as a strong cation acid resin such as Purolite C100CaMR, Amberlite IRP-69 and Dowex-50 can also be used. Particularly, the ion-exchange resin used is sodium polystyrene sulfonate (Amberlite IRP-69).

Non-permeable coatings of insoluble polymers, e.g., cellulose acetate, ethylcellulose, may be used as enteric coatings for delayed/modified release by inclusion of soluble pore formers in the coating, e.g., PEG, PVA, sugars, salts, detergents, triethyl citrate, triacetin, etc.

Suitable coating including ‘functional’ or ‘non-functional coating’ or ‘film-coating’ may comprise polymers like alkyl celluloses such as methyl or ethyl cellulose, hydroxyalkylcelluloses, hydroxypropylcellulose or hydroxypropyl methylcelluloses, hydroxyethyl cellulose, polyvinyl pyrrolidone, copolymer of vinyl pyrrolidone and vinyl acetate, polyvinylalcohol, polyethylene glycol, polyvinyl alcohol (PVA), acacia, corn, sucrose, gelatin, triacetin, shellac, cellulose acetate pthalate, lipids, synthetic resins, polymers based on methacrylic acid such as those marketed under the brand name of Eudragit®, dibutyl sebecate and diethyl tartrate, polysaccharides such as maltodextrin, opacifying agents like titanium dioxide and talc, and coloring agents. These may be applied from aqueous or non-aqueous systems or combinations of aqueous and non-aqueous systems as appropriate. Examples of such non-functional coats are commercially available Opadry® compositions. The coating may be present in an amount of about 0.1% w/w to about 50% w/w. In an embodiment, the coating according to the present invention is present in an amount of about 40% or less, 20% or less, e.g. 10% or less, 5% or less or 3% or less or 2% or less or 1% or less.

Having described the invention with reference to certain preferred embodiments, other embodiments will become apparent to one skilled in the art from consideration of the specification. The invention is further defined by reference to the following examples described in detail. It will be apparent to those skilled in the art that many modifications, both to materials and methods, may be practiced without departing from the scope of the invention.

The following examples (Ex.) are provided to illustrate embodiments of the disclosure but they are by no means intended to limit its scope.

### Example 1

TABLE 1

| Ingredients | Quantity (%) |
|---|---|
| Riociguat (Drug Substance) | 0.01-95 |
| Sugar Sphere/Microcrystalline cellulose sphere | 0-50 |
| (InertCore) | |
| Starch/Lactose/Microcrystalline Cellulose (Diluent) | 0-85 |
| Hydroxypropyl Cellulose/Hydroxypropyl methyl | 0-40 |
| Cellulose/Ethyl cellulose/Povidone/Methacrylate polymer/ | |
| Polyethylene oxide (Binder/Polymer) | |
| Sodium Starch Glycolate/Crospovidone/Croscarmellose | 0-40 |
| sodium (Disintegrant) | |
| Magnesium Stearate/Stearic acid (Lubricant) | 0-10 |
| Talc/Colloidal silicon di-oxide (Glidant) | 0-10 |
| Triethyl Citrate/PEG (Plasticizer) | 0-10 |
| Purified Water/Ethanol (Solvents) | q.s. |

Procedure: Extrusion-spheronization/Wet granulation/Fluidized bed coating.

### Examples 2-9

TABLE 2

| | Quantity (%) | | | | | | | |
|---|---|---|---|---|---|---|---|---|
| Ingredients | Ex. 2 | Ex. 3 | Ex. 4 | Ex. 5 | Ex. 6 | Ex. 7 | Ex. 8 | Ex. 9 |
| Riociguat | 0.01-80 | 0.01-50 | 0.01-30 | 0.01-20 | 0.01-10 | 0.01-5 | 0.01-5 | 0.01-5 |
| Microcrystalline | 0.1-70 | — | — | — | — | — | 0.1-80 | 0.1-80 |
| Cellulose | | | | | | | | |
| Lactose | — | 0.1-70 | 0.1-70 | 0.1-70 | 0.1-70 | 0.1-70 | — | — |
| Polyacrylate | 0.1-20 | — | — | — | — | — | — | — |
| dispersion 30% | | | | | | | | |
| Povidone | — | 0.1-20 | 0.1-20 | 0.1-20 | — | — | — | 0-20 |
| PEG 6000 | — | — | 0.1-20 | — | — | — | — | |
| Ethyl Cellulose | — | — | — | 0.1-20 | — | — | — | 10-40 |
| Hydroxypropyl | — | — | — | — | 0.1-20 | — | — | — |
| Cellulose | | | | | | | | |
| Hydroxypropyl | — | — | — | — | — | 0.1-20 | 20-40 | — |
| MethylCellulose | | | | | | | | |
| Magnesium Stearate | 0.01-5 | 0.01-5 | 0.01-5 | 0.01-5 | 0.01-5 | 0.01-5 | 0.01-5 | 0.01-5 |
| Talc/Colloidal | 0-5 | 0.01-5 | 0.01-3 | 0.01-2 | 0.01-1 | 0.01-1 | — | — |
| silicon dioxide | | | | | | | | |
| Purified water | q.s. | q.s. | q.s. | q.s. | q.s. | q.s. | q.s. | q.s. |

Procedure: Riociguat and suitable diluent (microcrystalline cellulose, lactose), suitable binder (optional) and release controlling polymer (optional) were sifted through suitable sieve and mixed. The blend was granulated with suitable solvent (water or polyacrylate dispersion). The granules were dried and sifted through suitable sieve. The lubricant (magnesium stearate) was sifted through suitable sieve and mixed with granules. The blend was compressed into tablets or filled into capsules.

### Example 10

TABLE 3

| Ingredients | Quantity (% w/w) |
|---|---|
| Riociguat | 1.5 |
| Hydroxypropyl Methyl | 30.0 |
| Cellulose (HPMC) | |
| Microcrystalline cellulose | 62.5 |
| Povidone | 5.0 |
| Magnesium Stearate | 1.0 |
| Purified water | q.s. |

Procedure: 1) Riociguat and povidone were mixed. 2) Blend of step 1) was mixed with hydroxypropyl methyl cellulose. 3) Blend of step 2) was mixed with microcrystalline cellulose. 4) The blend of step 3) was granulated with purified water. 5) The granules of step 4) were dried at 60° C.±10° C. for a suitable time period. 6) The dried granule of step 5) were sifted through a suitable sieve and blended with magnesium stearate. 7) The blend of step 6) was compressed into tablets or filled into capsules.

Results: The results of assay and dissolution profile of the above formulations are presented in below table 4. The dissolution profile of compositions prepared using quantitative compositions (Examples 10), as given below was measured in 900 ml of 6.8 phosphate buffer and 0.1% sodium lauryl sulfate (SLS) using a USP II apparatus (Paddle) at a temperature of 37±0.5° C. and a rotation speed of 75 revolutions per minute.

TABLE 4

| Time Point | % Drug release |
|---|---|
| (hrs.) | Ex. 10 |
| 1 | 4 |
| 2 | 7 |
| 3 | 9 |
| 4 | 12 |
| 6 | 18 |
| 8 | 24 |
| 10 | 31 |
| 12 | 40 |
| Assay (% w/w) | 100.0% |

The inventors have determined that certain excipients are critical for development of successful formulation with desired technical attributes. The present inventors have surprisingly found that in the extended release matrix formulation as represented in Example 10, the amount of release controlling polymer [such as HPMC] is critical, as demonstrated by some extended release formulations prepared with these excipient failing to achieve the desired technical attributes like release profile. For example, the inventors observed that for a successful extended release riociguat formulation, it is necessary to use the release controlling polymer in the range of about 25% to about 35% only, preferably less than 35%. The inventors determined that an amount of a release controlling polymer lower or higher than this range failed to achieve desired release profile. The dissolution profile of compositions prepared was measured in dissolution medium 900 ml of 6.8 phosphate buffer and 0.1% sodium lauryl sulfate (SLS) using a USP II apparatus (Paddle) at a temperature of 37±0.5° C. and a rotation speed of 75 revolutions per minute. The below-tabulated dissolution profile results present the dissolution of Example 10 modified to have 20% HPMC, 25% HPMC or 35% HPMC. Twenty percent HPMC provided a dissolution that was too rapid for an extended release dosage form while both 25% and 35% HPMC each provided a dissolution that is suitable for an extended release dosage form.

TABLE 5

| Time Point | % Drug release | | |
|---|---|---|---|
| (hrs.) | 20% HPMC | 25% HPMC | 35% HPMC |
| 1 | 66 | 49 | 7 |
| 2 | 86 | 65 | 10 |
| 3 | 94 | 76 | 13 |
| 4 | 97 | 84 | 15 |
| 6 | 98 | 92 | 21 |
| 8 | 98 | 94 | 28 |
| 10 | 99 | 95 | 35 |
| 12 | 98 | 96 | 42 |
| 16 | 98 | 95 | 50 |
| 20 | 98 | 96 | 64 |
| 24 | 98 | 96 | 88 |

Based on Example 10 and the results reported above at 25% and 35% HPMC in the formulation, the following release profile was determined for a once or twice daily orally administered, extended release formulation of riociguat that optionally is in the form of a matrix tablet:

| Time Point | |
|---|---|
| (hrs.) | % Drug release |
| 1 | 4-49 |
| 2 | 10-65 |
| 3 | 13-76 |
| 4 | 15-84 |
| 6 | 21-92 |
| 8 | 28-94 |
| 10 | 35-95 |
| 12 | 42-96 |
| 16 | 50-95 |
| 20 | 64-96 |
| 24 | 88-96 |

Such compositions would include the riociguat to the release controlling polymer, e.g., HPMC, at ratios of about 1:15 to about 1:25, about 1:16 to about 1:24, including a ratio of 1:20 for 30% w/w HPMC in the composition with 1.5% w/w riociguat.

### Coated Dosage Form:

TABLE 6

Quantity (%)

| | | | |
|---|---|---|---|
| Ingredients | Ex. 11 | Ex. 12 | Ex. 13 |

Core

| | | | |
|---|---|---|---|
| Riociguat | 0.01-50 | 0.01-10 | 0.01-5 |
| Starch | 0-70 | — | — |
| Microcrystalline | 0-70 | 0.1-70 | — |
| Cellulose | | | |
| Hydroxypropyl Cellulose | 0-70 | — | 0.1-70 |
| Sodium Starch Glycolate | 0-40 | 0.1-15 | 0.1-15 |
| Magnesium Stearate | 0.01-5 | 0.01-2 | 0.01-2 |

Coating

| | | | |
|---|---|---|---|
| Ethylcellulose | 0-30 | 0.1-15 | — |
| HPMC | 0-30 | — | 0.1-15 |
| Purified water | q.s. | q.s. | q.s. |

Procedure: Riociguat, starch, microcrystalline cellulose and hydroxypropylcellulose were sifted and mixed. The blend was granulated with sufficient quantity of purified water. The granules were dried, sifted and mixed with microcrystalline cellulose and sodium starch glycolate. The blend was mixed with magnesium stearate and compressed into tablets. Coating: HPMC based coating material was dissolved in sufficient quantity of purified water and mixed with ethylcellulose based aqueous coating material. The core tablets were coated with the coating solution to a desired weight gain.

Multi-Particulate Dosage Form:

TABLE 7

| | Example | | |
|---|---|---|---|
| Composition | Ex. 14 | Ex. 15 | Ex. 16 |

| | | |
|---|---|---|
| S. No. | Ingredients | % w/w |

Inert Core

| | | | | |
|---|---|---|---|---|
| 1. | Microcrystalline cellulose | 1-50 | — | 1-50 |
| | sphere | | | |
| 2. | Sugar spheres | — | 1-50 | — |

Seal coat

| | | | | |
|---|---|---|---|---|
| 3. | Hydroxypropyl methylcellulose | 0.1-2 | — | — |
| 4. | Ethyl cellulose | — | — | 0.1-2 |

Drug Layer

| | | | | |
|---|---|---|---|---|
| 5. | Riociguat | 0.01-70 | 0.01-70 | 0.01-70 |
| 6. | Povidone/Hypromellose | 0.1-5 | 0.1-5 | 0.1-5 |
| 7. | Crospovidone | — | 1-20 | 1-20 |
| 8. | Purified Water | q.s | q.s | q.s |

Seal Coating

| | | | | |
|---|---|---|---|---|
| 9. | Hypromellose | 0.1-5 | — | 0.1-5 |
| 10. | Talc | 0.5-5 | — | — |
| 11. | Titanium dioxide | 0.1-2 | 0.1-2 | 0.1-2 |
| 12. | Polysorbate 80 | 0.1-2 | 0.1-2 | — |
| 13. | Purified water | q.s | q.s | q.s |

Extended release Layer

| | | | | |
|---|---|---|---|---|
| 14. | Methacrylate polymer | 0.5-5 | 0.5-5 | 0.5-5 |
| 15. | Talc | 0.5-2 | 0.5-2 | 0.5-2 |
| 16. | Triethyl Citrate | 0.5-2 | 0.5-2 | 0.5-2 |
| 17. | Sodium lauryl sulfate | — | 0.1-2 | 0.1-2 |
| 18. | Purified Water/solvents | q.s. | q.s. | q.s. |

Outer layer

| | | | | |
|---|---|---|---|---|
| 19. | Hypromellose/Povidone | 0.1-20 | 0.1-20 | 0.1-20 |
| 20. | Purified water | q.s | q.s | q.s |

Preferred method of manufacture: Fluidized bed Pelletization (coating the inert core with a drug layer comprising riociguat, optionally applying the seal coating of a water soluble/insoluble polymer on the drug coated core, coating the resultant product with extended release coat comprising a release controlling polymer and at least one or more pharmaceutically acceptable excipient, optionally applying a seal coat on the extended release coated core and applying outer coat on the core).

TABLE 8

| | Example | | | |
|---|---|---|---|---|
| Composition | Ex. 17 | Ex. 18 | Ex. 19 | Ex. 20 |

| | | |
|---|---|---|
| S. No. | Ingredients | % w/w |

Drug Layered Pellets

| | | | | | |
|---|---|---|---|---|---|
| 1. | Riociguat | 0.01-10 | 0.92 | 0.97 | 0.97 |
| 2. | Povidone | 0-20 | 1.84 | 0.97 | 0.97 |
| 3. | MCC Spheres | 10-95 | 88.15 | 93.29 | — |
| 4. | Sugar Spheres | 10-95 | — | — | 93.29 |
| 5. | Methanol | q.s. | q.s | q.s | q.s |

Extended Release Pellets

| | | | | | |
|---|---|---|---|---|---|
| 6. | Ethyl Cellulose 10 | 0-30 | 9.09 | — | — |
| | CPS | | | | |
| 7. | Ethyl Cellulose 7 | 0-30 | — | 9.09 | 4.76 |
| | CPS | | | | |
| 8. | Methanol | q.s. | q.s | q.s | q.s |

Extragranular component

| | | | | | |
|---|---|---|---|---|---|
| 9. | Microcrystalline | 0-60 | 20.83 | 21.29 | 23.33 |
| | cellulose | | | | |
| 10. | Mannitol and | 0-60 | 27.78 | 27.78 | 27.78 |
| | Maize starch | | | | |
| 11. | Crospovidone | 0-20 | 5.56 | 5.56 | 5.56 |
| 12. | Magnesium Stearate | 0-5 | 0.46 | 0.46 | 0.46 |

Procedure: 1) MCC spheres/sugar spheres were optionally coated with a water-soluble seal or seal coating layer consisting essentially of water-soluble polymers, 2) Drug solution was prepared by adding povidone and riociguat in methanol and stirred until clear solution is obtained, 3) Drug solution of step 2) was sprayed over MCC spheres/sugar spheres to obtain the desired weight gain, 4) The drug layered pellets of step 3) were optionally coated with a water-soluble seal or seal coating layer consisting essentially of water-soluble polymers, 5) Extended release coating solution was prepared by dissolving ethyl cellulose in methanol and stirred until clear solution formed, 6) The drug layered pellets of step 3) or 4) were coated with the extended release coating solution of step 5) to obtain the desired weight gain, 7) Microcrystalline cellulose, mannitol, maize starch and crospovidone were co-sifted through a suitable mesh, 8) Extended release pellets of step 6) were blended with the extragranular material of step 7). 9) Magnesium stearate was sifted through a suitable mesh and blended with the material of step 8). 10) The blend of step 9) was compressed into tablets or filled into capsules.

The dissolution profile of compositions prepared was measured in 900 ml of 6.8 phosphate buffer and 0.1% sodium lauryl sulfate (SLS) using a USP II apparatus (Paddle) at a temperature of 37±0.5° C. and a rotation speed of 100 revolutions per minute.

TABLE 9

| Time Point | % Drug release | | |
|---|---|---|---|
| (hrs.) | Ex. 18 | Ex. 19 | Ex. 20 |
| 1 | 15 | 8 | 67 |
| 2 | 17 | 10 | 75 |
| 3 | 19 | 11 | 79 |
| 4 | 20 | 13 | 82 |
| 6 | 22 | 15 | 85 |
| 8 | 24 | 16 | 86 |
| 10 | 25 | 16 | 87 |
| 12 | 27 | 17 | 88 |

What is claimed:

1. An extended release pharmaceutical composition suitable for once or twice daily dosing comprising:
a) riociguat in an amount of about 0.01% to about 10% by weight,
b) a release controlling material selected from the group consisting of hydrophilic release controlling materials and hydrophobic release controlling materials or combinations thereof in an amount of about 25% to about 35% by weight; and
c) one or more other pharmaceutically acceptable excipients,
wherein the composition is free of glidant and the weight ratio of riociguat to release controlling material in the composition is more than 1:15.

2. The pharmaceutical composition according to claim 1, wherein the hydrophilic release controlling material is selected from the group consisting of hydroxypropyl methylcellulose, hydroxypropyl cellulose, povidone, polyethylene glycols, vinyl acetate copolymers, polysaccharides as alginates, xanthan gum, chitosan, carrageenan, dextran, polyalkylene oxides as polyethylene oxide, methacrylic acid copolymers, maleic anhydride/methyl vinyl ether copolymers, and carbomers.

3. The pharmaceutical composition according to claim 1, wherein the hydrophobic release controlling material is selected from the group consisting of ethyl cellulose, cellulose acetate, polyvinyl acetate dispersion, cellulose acetate phthalate, cellulose triacetate, poly (methyl methacrylate), poly (ethyl methacrylate), poly (butyl methacrylate), poly (isobutyl methacrylate), poly (hexyl methacrylate), poly (isodecyl methacrylate), poly (lauryl methacrylate), poly (phenyl methacrylate), poly (methyl acrylate), poly (isopropyl acrylate), poly (isobutyl acrylate), poly (octadecyl acrylate), beeswax, carnauba wax, paraffin wax, cetostearyl alcohol, stearyl alcohol, cetyl alcohol, glyceryl monostearate, glycerol monooleate, acetylated monoglycerides, glyceryl behenate and hydrogenated vegetable oils.

4. The pharmaceutical composition according to claim 1, wherein the one or more other pharmaceutically acceptable excipients are selected from the group consisting of diluent, binder, disintegrant, antioxidant, surfactant, plasticizer, stabilizer, anticaking agent, anti-foaming agent, lubricant, film-forming polymer, opacifier and coloring agent.

5. The pharmaceutical composition according to claim 4, wherein the diluent is selected from the group consisting of lactose, cellulose, microcrystalline cellulose, mannitol, calcium phosphate, starch, pregelatinized starch, and the like, used either alone or in combination thereof.

6. The pharmaceutical composition according to claim 4, wherein the binder is selected from the group consisting of polyvinylpyrrolidone, cellulose derivatives such as hydroxypropyl methylcellulose, hydroxypropyl cellulose, methacrylic acid polymers, and acrylic acid polymers.

7. The pharmaceutical composition according to claim 1, wherein the composition is in the form of granules, tablets, pellets, and capsules.

8. The pharmaceutical composition according to claim 7, wherein the tablet composition is a matrix tablet dosage form.

9. The pharmaceutical composition according to claim 7, wherein the tablet composition is a multiparticulate tablet or capsule dosage form.

10. The pharmaceutical composition according to claim 1, wherein the weight ratio of riociguat to release controlling material in the composition is between about 1:15 to about 1:25.

11. The pharmaceutical composition according to claim 1, wherein the composition comprises riociguat in an amount from about 0.5 mg to about 7.5 mg.

12. The pharmaceutical composition according to claim 1, wherein the composition is free of colloidal silicon dioxide as glidant.

13. The pharmaceutical composition according to claim 1, wherein the composition free of any swelling agent selected from polyethylene oxide derivatives and/or carbopol derivatives.

14. The pharmaceutical composition according to claim 1, the weight ratio of riociguat to release controlling material in the composition is about 1:20.

15. The pharmaceutical composition according to claim 1, wherein the ratio of riociguat to diluent in the composition is at least 1:30.

16. The pharmaceutical composition according to claim 1, wherein the ratio of riociguat to binder in the composition is less than 1:5.

17. The pharmaceutical composition according to claim 1, wherein particle size distribution of riociguat is D90 is less than about 50 μm.

18. The pharmaceutical composition according to claim 1, wherein the composition is prepared by wet granulation.

19. An extended release pharmaceutical tablet composition comprising:
a) riociguat in an amount of about 0.01% to about 10% by weight,
b) a release controlling material selected from the group consisting of hydroxypropyl methylcellulose, hydroxypropyl cellulose, methacrylic acid copolymers, ethyl cellulose, cellulose acetate or combination thereof in an amount of about 25% to about 35% by weight,
c) a diluent selected from the group consisting of lactose, cellulose, microcrystalline cellulose, mannitol, starch, pregelatinized starch, or combination thereof in an amount of about 30% to about 80% by weight; and
d) a binder selected from the group consisting of polyvinylpyrrolidone, hydroxypropyl methylcellulose, hydroxypropyl cellulose, or combination thereof in an amount of about 2% to about 10% by weight,
wherein the composition is free of any glidant and the ratio of riociguat to release controlling material in the composition is more than 1:15.

20. An extended release pharmaceutical matrix tablet consisting of:
a) riociguat in an amount of about 0.01% to about 10% by weight;
b) one or more release controlling materials selected from hydroxypropyl methylcellulose and ethyl cellulose in an amount of about 25% to about 35% by weight, wherein the weight ratio of riociguat to release controlling material in the tablet is between about 1:15 to about 1:25;
c) microcrystalline cellulose in an amount of at least about 25% by weight;
d) polyvinylpyrrolidone in an amount of at least about 1% by weight; and
e) magnesium stearate in an amount of at least about 0.1% by weight,
wherein the tablet exhibits about 4-49% drug release in about 1 hour, about 10-65% drug release in about 2 hours, about 13-76% drug release in about 3 hours, about 15-84% drug release in about 4 hours, about 21-92% drug release in about 6 hours, about 28-94% drug release in about 8 hours, about 35-95% drug release in about 10 hours, about 42-96% drug release in about 12 hours, about 50-95% drug release in about 16 hours, about 64-96% drug release in about 20 hours, and about 88-96% drug release in about 24 hours, when measured in 900 ml of 6.8 phosphate buffer and 0.1% sodium lauryl sulfate (SLS) using a USP II apparatus (Paddle) at a temperature of 37±0.5° C. and a rotation speed of 75 revolutions per minute.
